(19) United States
(12) Patent Application Publication
(10) Pub. No.: US 2002/0111382 A1
(43) Pub. Date: Aug. 15, 2002

(54) Antiviral compounds and method of treating viral infections

(76) Inventors: Xiangmin Cui, Cumberland, RI (US); Qiang Xu, Cupertino, CA (US); Long-Ze Lin, East Providence, RI (US); Lianyun Zhao, East Providence, RI (US); Shunxiang Zhang, Chapel Hill, NC (US); Lydia Wu, San Francisco, CA (US); Yuefeng Lu, San Carlos, CA (US)

Correspondence Address: SCHNADER HARRISON SEGAL & LEWIS, LLP, 1600 MARKET STREET, SUITE 3600, PHILADELPHIA, PA, 19103

(21) Appl. No.: 09/948,312

(22) Filed: Sep. 6, 2001

Related U.S. Application Data
(60) Provisional application No. 60/230,468, filed on Sep. 6, 2000.

(51) Int. Cl. A61K 31/225; A61K 31/216; C07C 69/66; C07C 69/76

(52) U.S. Cl. 514/533; 514/547; 560/104; 560/106; 560/180

(57) ABSTRACT

Disclosed is method for treating viral infections and compounds for use in such treatments. The method involves administering a pharmaceutical formulation including 3,5-dicaffeoylquinic acid or its analogs or derivatives to a patient. The derivative or analog compounds are presented in a pharmaceutical formulation comprising a substantially pure compound of formula I:

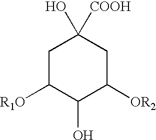

wherein R₁ and R₂ are selected from the group consisting of the following moieties:

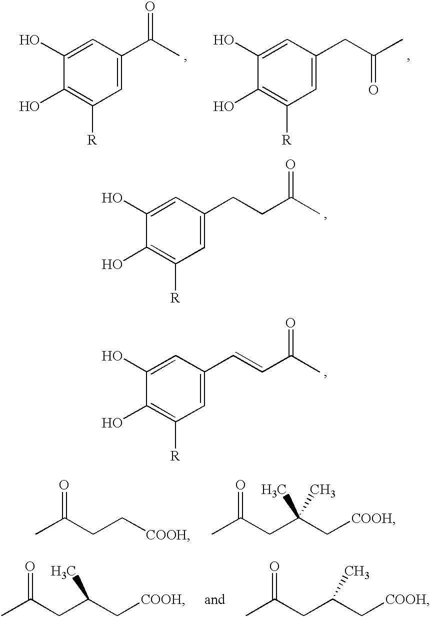

where R is H or a halogen.

This application claims priority to U.S. Provisional Patent Application No. 60/230,468 filed Sep. 6, 2000.

### FIELD OF THE INVENTION

The present invention pertains to a method for treating viral infections, specifically infections caused by respiratory syncytial virus and parainfluenza, and compounds useful for such treatment. More specifically, the invention pertains to analogs or derivatives of 3,5-dicaffeoylquinic acid and a method of treating viral infections by administration of a pharmaceutical formulation comprising substantially pure 3,5-dicaffeoylquinic acid or its analogs or derivatives, in a suitable pharmaceutically acceptable carrier.

### DESCRIPTION OF THE RELATED ART

Shuang Huang Lian is a traditional Chinese medicine commonly used to treat acute respiratory infections (ARI). Clinical data indicate that Shuang Huang Lian is effective in alleviating symptoms of ARIs, shortening the duration and severity of the disease, and minimizing potential long-term consequences of lung infection. In a randomized single blind clinical trial for the treatment of acute bronchiolitis (96 patients) Shuang Huang Lian showed greater potency than the antibiotics lincomycin and cephazolin. The main outcomes, assessed blindly, were symptomatic improvement in cough, fever, wheezing, and chest crackles. There were no adverse effects of Shuang Huang Lian treatment reported.

Shuang Huang Lian is a combination of three Chinese medical plants, Lonicera japonica Thunb, Scutellaria amoena C. H. Wright, and Forsythia suspense Thunb. However, it may not be necessary to combine all three. Despite its common usage, the exact mechanism of action of Shuang Huang Lian is unclear. Heretofore, the active molecule has been unknown. We have now discovered the active compound in this traditional medicine treatment.

### SUMMARY OF THE INVENTION

The present invention comprises analogs or derivatives of 3,5-dicaffeoylquinic acid and a method of treating viral infections by administration of a pharmaceutical formulation comprising substantially pure 3,5-dicaffeoylquinic acid or its analogs or derivatives, in a suitable pharmaceutically acceptable carrier.

### BRIEF DESCRIPTION OF THE DRAWINGS

FIG. 1 is a graph showing inhibitory activity of L001 extract against RSV in vitro.

FIG. 2 is a graph showing inhibitory activity of F001 extract against RSV in vitro.

FIG. 3 is a graph showing inhibitory activity of S001 extract against RSV in vitro.

FIG. 4 is a flowchart depicting the reverse-phase HPLC methodology employed in fractionating L001.

FIGS. 5a-5c are graphs showing cell viability versus DCQA without virus.

FIGS. 5d-5f are graphs showing %CPE reduction in cells infected with RSV and treated with DCQA.

### DETAILED DESCRIPTION

Initially, the three separate plant components of the traditional Chinese medicine Shuang Huang Lian were studied. Extracts were made and tested from each plant as follows.

Dried and powdered buds of Lonicera japonica Thunb, roots of Scutellaria amoena C. H. Wright, and fruit of Forsythia suspensa Thunb were extracted with 70% acetone three times. The solution was concentrated to small volume and then submitted to DM-130 column eluting with water, 50% acetone/50% water, and 100% acetone respectively. The fractions from 50% acetone elution were evaporated under vacuum. The extracts were assigned the designations L001, S001, and F001 respectively.

To determine the antiviral activities of the crude extracts from the three fractions, the CPE (virus-induced cytopathogenic effects) inhibition assay was employed. The assay was conducted using the following cell lines:

| Cell line | Virus Type | Cells/0.1 ml/well |
|---|---|---|
| Hep-2 | RSV | 10,000 |
| Vero | RSV | 10,000 |

Plant extracts were diluted serially, with a high testing concentration starting at 1000 μg/ml. Microbial contamination in the dilutions was controlled by the addition of penicillin/streptomycin to the cell culture media.

The cells (1×104 cells/0.1 ml/well) were pre-grown overnight at 37° C. in 96-well tissue culture plates using EMEM (Eagle's Minimum Essential Medium—available from Sigma of St. Louis, Mo.) supplemented with 10% heat inactivated fetal bovine serum (FBS) and penicillin/streptomycin (50 μg/ml). Antiviral assays were designed to test up to six concentrations of extracts/fractions/molecules in triplicate against the virus in microplate wells containing the host cell monolayers. The positive control for the assay was Ribavirin.

Prior to the addition of drug and virus to the 96-well plated, cells were washed with phosphate buffered saline, and 0.1 ml of extracts/fractions/molecules and 0.1 ml of the virus suspension were mixed and incubated at 37° C. for 1 hour. The 0.2 ml of incubated virus/drug suspension was added to the cell culture. Maximum CPE was generally observed on day 5 in the untreated virus control culture.

Cell controls containing media alone, virus-infected controls containing medium and virus, and drug cytotoxicity controls containing media and each drug concentration were run simultaneously with the test samples. CPE inhibition was determined by a dye (MTS) uptake procedure. This method measures the cell viability and is based on the reduction of the Tetrazolium MTS by mitochondrial enzymes of viable host cells to MTS formazan. In this method, 50 μl of MTS and PMS (an electron coupling agent) are added to each of the plate wells. The plates are incubated at 37° C. for 4 hours. The orange color or the MTS formazan is then measured at 490 nm. The optical density is a function of the amount of formazan produced, which is proportional to the number of viable cells.

A computer program was used to calculate the percentage of CPE reduction of the virus-infected wells and the percent cell viability of uninfected drug control wells. While the details of the calculation are not critical, importantly, the calculations were uniform in that the same algorithm was used for all calculations.

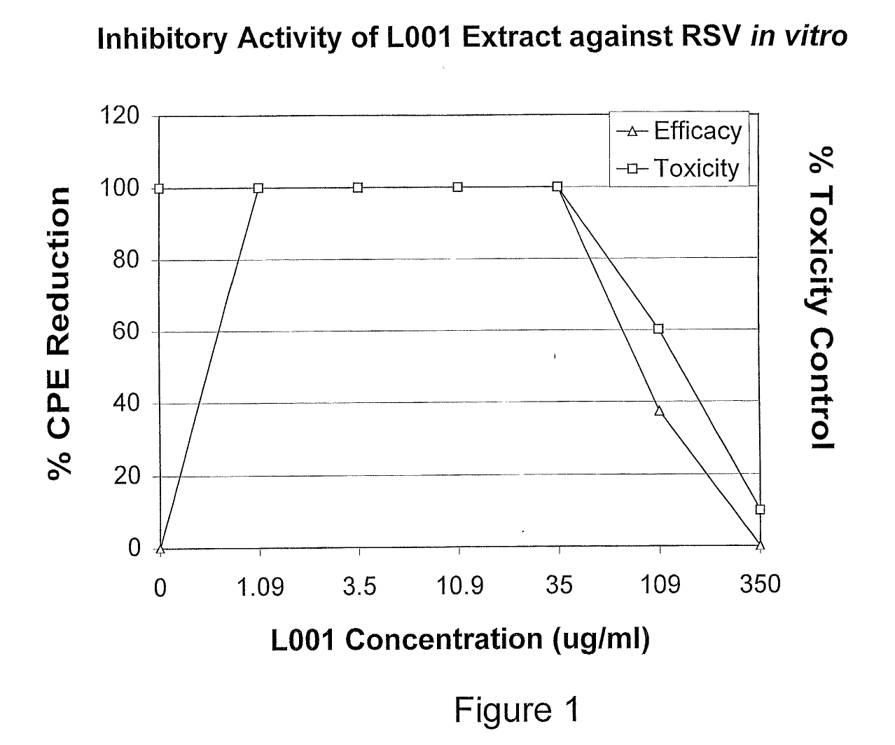

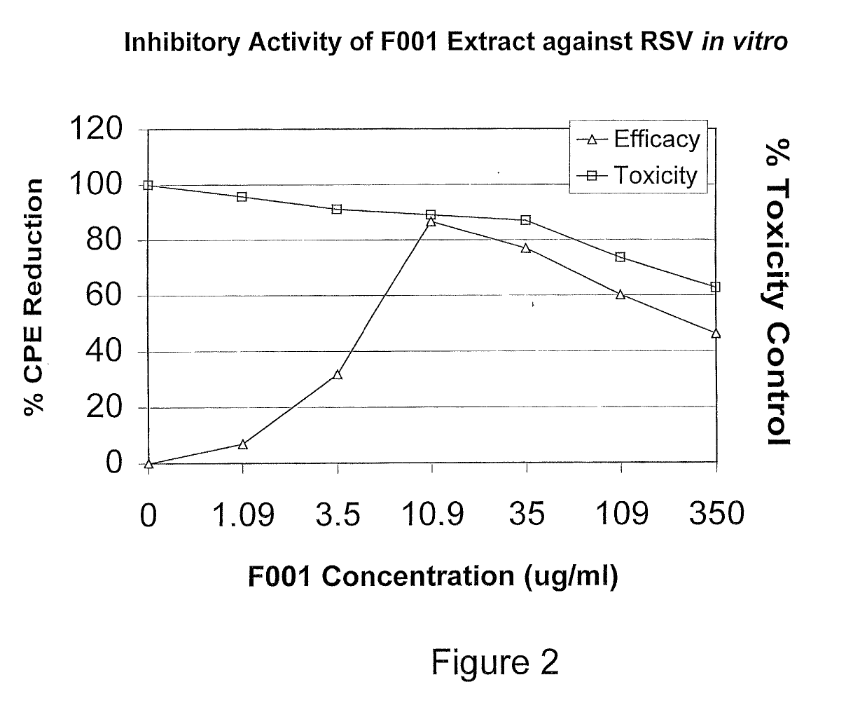

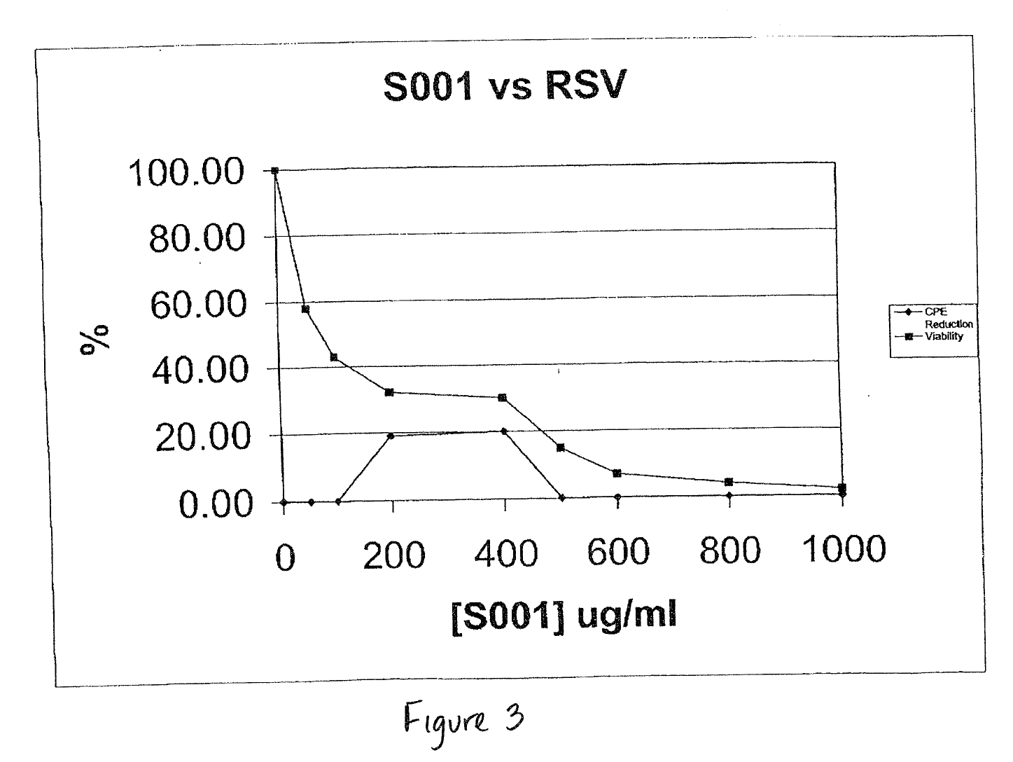

As can be seen from FIGS. 1 and 2, L001 and F001 demonstrated very potent anti-RSV activities in the cell-based assay. Both extracts show more potent activity than the positive control Ribivarin, used in these experiments. In contrast, there was no significant anti-RSV activity observed for extract S001, as shown in FIG. 3.

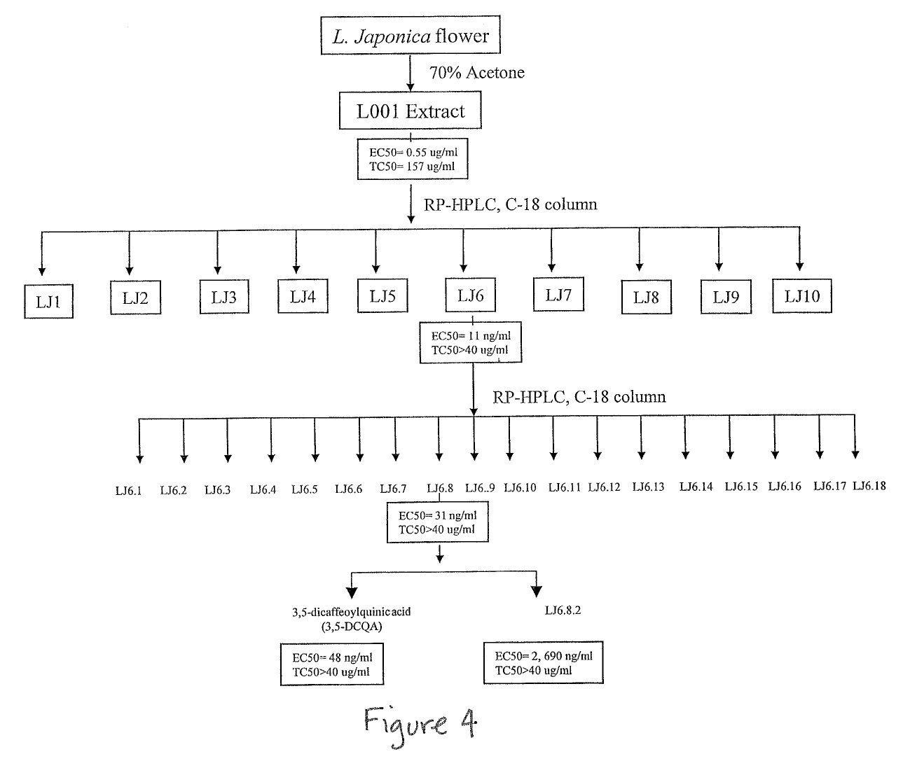

We have now fractionated L001 using reverse-phase HPLC and have thereby discovered several compounds with pronounced antiviral activity. The methodology for the reverse-phase HPLC is shown in FIG. 4. It has now been found that the compounds of Formula I have potent anti-viral activity. These compounds are useful as anti-viral agents for administration to patients afflicted with viral infections.

The compounds of the present invention have the formula I

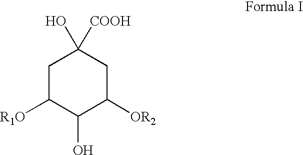

where R1 and R2 are selected from the group consisting of the following moieties.

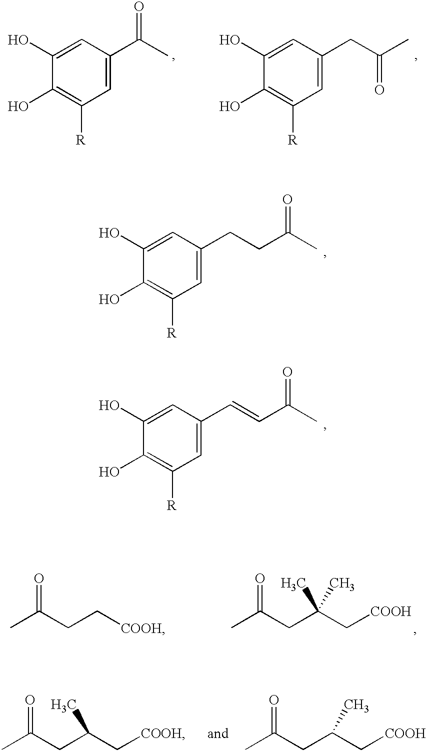

where R is H or a halogen. These compounds are useful for treating viral infections when administered in a substantially pure form. By “substantially pure”, it is meant that the compound is either synthesized or purified from a naturally occurring state, such that, if naturally occurring, the compound has only trace quantities of materials that occur with it in a natural state. Preferably, such other materials are present only in an amount whereby no effect on the patient can be observed by virtue of their inclusion in a composition containing the substantially pure compound that is administered to that patient.

In a method according to the invention, a pharmaceutical formulation comprising a substantial pure compound of formula I and a pharmaceutically acceptable carrier or other agents, is administered to a patient having a viral infection. Those skilled in the art will recognize that the formulation can take many forms, including solid, liquid, liquid filled capsule, etc for administration orally or parenterally. Any current or later developed method of administration can be employed, including extended release and other types of formulations and dosage forms.

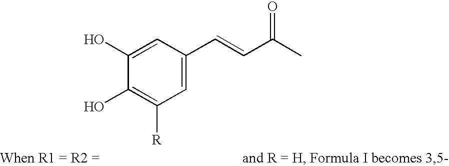

dicaffeoylquinic acid (DCQA), which is shown as formula Ia.

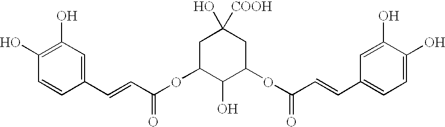

### Formula Ia—3,5-dicaffeoylquinic acid

This particular compound was tested using the following methods.

Cells and Virus

Both HEp-2 (human epithelial carcinoma) and the respiratory syncytial virus (RSV, A2 strain) used in the present study were obtained originally from the American Type Culture Collection (ATCC, Manassas, Va.). HEp-2 cells were passaged using Eagle's minimum essential medium (EMEM) supplemented with fetal bovine serum (FBS), penicillin (100 units/ml), and streptomycin (100 μg/ml) at 37° C. and 5% CO2.

Stock of RSV was prepared by infecting flasks of HEp-2 cells that had less than 10 passages. When the monolayers in these flasks exhibited approximately 80-90% syncytial formation, the remaining adherent cells were released using cell scrapper (Nalge Nunc International). Afterward, the cells and medium were collected, pooled, and clarified by centrifugation. The clarified supernatant was aliquoted, frozen in ethanol bath, and stored at gas phase in a cryogenic unit until analysis.

Cytopathogenic Effect Reduction Assay

A virus-induced cytopathogenic effects (CPE) inhibition assay was employed to evaluate antiviral activity of DCQA against RSV in HEp2 cells.

The cells seeded at 1.0×104 cells/0.1 ml/well were pre-grown overnight at 37° C. and 5% CO2 in 96-well tissue culture plates (Coming Inc, Coming, N.Y.) using EMEM supplemented 10% FBS, penicillin (100 units/ml), and streptomycin (100 μg/ml). After overnight growth, medium was removed and cells were washed once with PBS prior to addition of 0.1-ml mixture of properly diluted virus and/or DCQA in EMEM, 2% heat-inactivated FBS, penicillin (100 units/ml), and streptomycin (100 μg/ml). To determine the effective dose of DCQA, a series of DCQA dilutions were used with a starting level at 40 μg/ml and a final concentration at 0.0128 μg/ml. Cell controls containing medium alone, virus-infected controls containing medium and virus, and the drug cytotoxicity controls containing medium and each drug concentration were run simultaneously with the test samples. The plates were incubated at 37° C. in a humidified atmosphere containing 5% CO2 until maximum CPE was observed in the untreated virus control cultures (Day 5).

CPE inhibition was determined by a MTS assay (Promega, Madison, Wis.). Loss of cell viability was quantified by the decrease in the ability of cells to metabolize the dye 3-(4,5-dimethylthiazol-2-yl)-5-(3-carboxymethoxyphenyl)-2-(4-sulfophenyl)-2H-tetrozolium (MTS) in the presence of an electron coupling reagent phenazine methosulfate (PMS). In this system, MTS is bioreduced by mitochondrial enzymes of living cells into formazan. The quality of formazan product when spectrophotometrically measured at 490 nm is directionally proportional to the number of living cells in culture. Twenty μl of MTS and PMS mixtures at a final concentration of 333 μg/ml and 25 μM, respectively, was added to each of the plate wells. The plates were incubated at 37° C. for 2 hours before optical density at 490 nm was measured using Spectramax 190 (Molecular Devices, Sunnyvale, Calif.). A computer program was utilized to calculate the percent of CPE reduction of the virus-infected wells, the percent cell viability of uninfected drug control wells, and the minimum effective DCQA concentration that reduced the CPE by 50% (EC50).

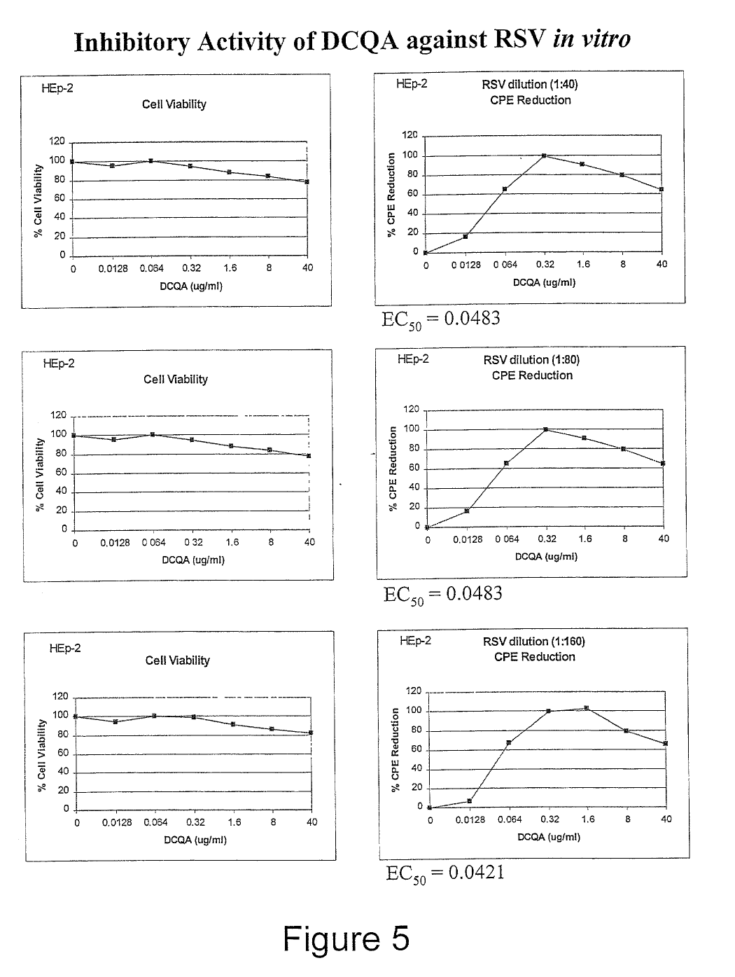

The results of the CPE study are shown in FIG. 5, where the left column of graphs shows cell viability versus DCQA added, without virus, and the right column shows %CPE reduction for cells infected with RSV and treated with DCQA. For each chart showing % CPE reduction, the EC₅₀ is shown, which is the concentration of DCQA needed to give a 50% reduction in virus-induced cytopathogenic effects.

We claim:

1. A method for treating viral infections comprising administering to a patient a pharmaceutical formulation comprising a substantially pure compound of the following formula: 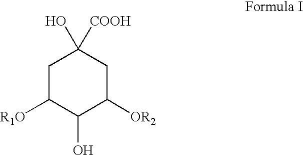
wherein R₁ and R₂ are selected from the group consisting of the following moieties: 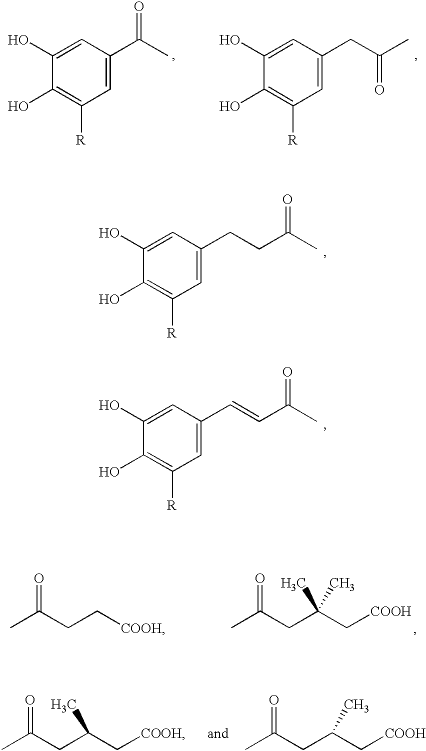
where R is H or a halogen.

2. The method according to claim 1 wherein said pharmaceutical formulation is administered in an amount effective to reduce virus-induced cytopathogenic effects.

3. The method according to claim 1 wherein said administration is oral.

4. The method according to claim 1 wherein said administration is parenteral.

5. The method according to claim 1 wherein said pharmaceutical formulation further comprises at least one pharmaceutically acceptable agent selected from a carrier, a coating, an excipient, adjuvant, filler, and binder.

6. An antiviral compound of the following formula: 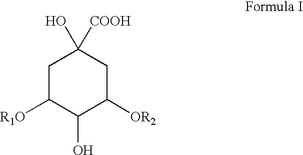
wherein R₁ and R₂ are selected from the group consisting of the following moieties: 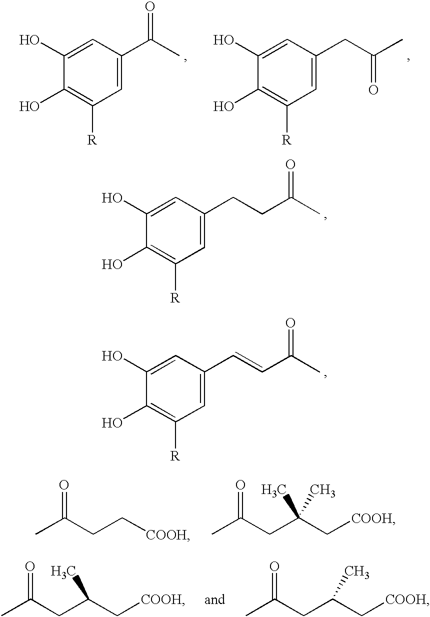
where R is H or a halogen, providing that both R₁ and R₂ cannot both be

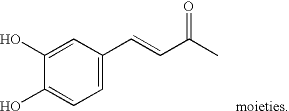

7. A pharmaceutical formulation comprising:
an effective amount of an antiviral compound of the following formula: 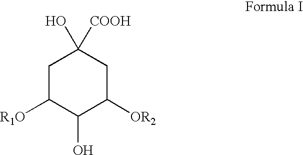
wherein R₁ and R₂ are selected from the group consisting of the following moieties: 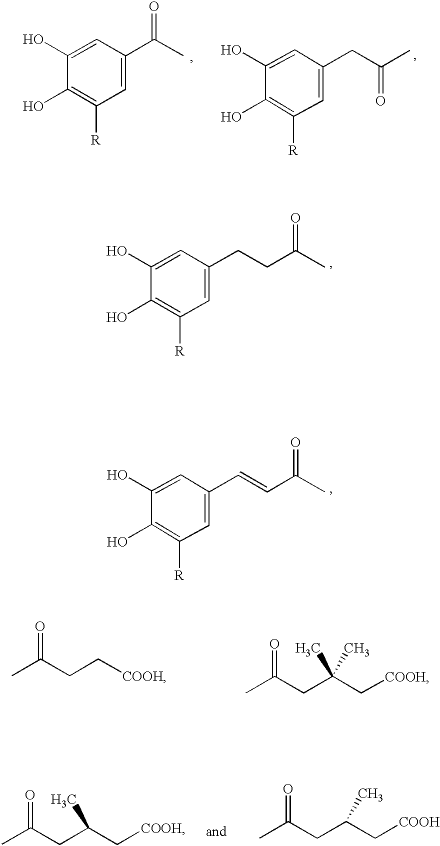
where R is H or a halogen, providing that both R₁ and R₂ cannot both be 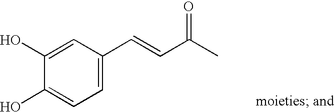
one or more pharmaceutically acceptable agent selected from the group consisting of a carrier, a diluent, an excipient, and an adjuvant.
